(19) United States
(12) Patent Application Publication
(10) Pub. No.: US 2010/0240724 A1
(43) Pub. Date: Sep. 23, 2010

(54) Edible Film

(75) Inventors: Seok Hoon Chang, Seoul (KR); Kyoung Tae Jung, Seoul (KR)

Correspondence Address: DOWELL & DOWELL P.C., 103 Oronoco St., Suite 220, Alexandria, VA, 22314, US

(73) Assignee: C.L. PHARM, Seoul (KR)

(21) Appl. No.: 12/680,720

(22) PCT Filed: Sep. 25, 2008
(86) PCT No.: PCT/KR08/05698

§ 371 Date: Mar. 29, 2010

(30) Foreign Application Priority Data
Oct. 3, 2007 (KR) 10-2007-0099559
Sep. 23, 2008 (KR) 10-2008-0093403

(51) Int. Cl. A61K 31/4184; A61K 47/42; A61K 31/4045; A61P 1/00; A61P 3/00; A61P 5/00; A61P 15/00; A61P 19/00; A61P 21/00; A61P 25/00; A61P 29/00; A61P 31/12; A61P 35/00

(52) U.S. Cl. 514/394; 514/774; 514/418

(57) ABSTRACT

The present invention relates to an edible film composition comprising a waxy starch hydrolysate, a modified starch and a water-soluble polymer, and an edible film comprising the same. The present edible film may not only have an excellent film forming property, solubility and feeling in the oral cavity, but also be easily prepared at low cost, and thus be usefully used as an oral cleanser or a bad breath remover for oral cleansing, and a soluble formulation on tongue for uptake of drug in the oral cavity.

### TECHNICAL FIELD

The present invention relates to an edible film composition and an edible film comprising a waxy starch hydrolysate, a modified starch and a water-soluble polymer.

### BACKGROUND ART

Generally, an edible film is based on a water-soluble polymer such as pullulan, sodium alginate, pectin, gelatin, carrageenan, xanthan gum and locust bean gum, etc., and prepared by ailing a plasticizer, an emulsifier, a sweetener, an acidulant, a flavoring and other additives thereto.

US Patent Application Publication No. 2005/0031675 discloses a soluble edible film using pullulan as a film forming material, and U.S. Pat. No. 6,656,493 discloses a water-soluble edible film comprising sodium alginate and maltodextrin. U.S. Pat. No. 6,709,669, U.S. Pat. No. 7,132,113 and U.S. Pat. No. 6,419,903 disclose a soluble edible film prepared by using gelatin, an edible film comprising hydroxypropyl cellulose and a modified starch and an edible film comprising hydroxypropylmethyl cellulose and a pregelatinized starch, respectively.

These conventional techniques disclose edible films consisting of water-soluble polymer materials. However, since the water soluble polymers have excellent film formability, but are expensive, not smoothly supplied and have a slow solubility rate in the oral cavity, they had a problem for use as an oral cleanser or a drug delivery system for oral administration.

To overcome problems of soluble edible films according to such conventional techniques, U.S. Pat. No. 6,528,088 discloses a film comprising a starch and a plasticizer, and JP Patent Application Publication No. 2005306960 discloses a soluble edible film comprising a modified high amylose starch and a modified normal starch, and a gelling agent.

However, the edible film in U.S. Pat. No. 6,528,088 as described above uses a large quantity of plasticizers with high molecular weight starches and modified starches, and thus has an excessive foreign material sensation. In addition, the edible film in JP Patent Application Publication No. 2005306960 has excellent film formability by using a substrate for film forming, wherein a gelling agent or a plasticizer is added to a mixture of a modified high amylose starch and a modified and etherified starch or maltodextrin. However, it uses an excessive amount of high molecular weight materials, and thus has the impaired solubility in the oral cavity and the remarkable feeling of slipperiness.

Furthermore, starches and modified starches used in U.S. Pat. No. 6,528,088 and JP Patent Application Publication No. 2005306960 as described above are not only aged in an aqueous solution at normal temperature to cause discomfort in work, but if they are stored in a tank for a long time, gel formed by aging closes a filter mesh or is included in the prepared film to cause a problem in quality of film.

### DISCLOSURE OF INVENTION

### Technical Problem

The present invention is intended to provide an edible film composition which may have an excellent film forming property, with being even orally dissolved in a short time, and an excellent feeling in oral cavity, and be prepared at low cost, and an edible film comprising the same.

### Technical Solution

The present invention provides an edible film composition comprising a waxy starch hydrolysate, a modified starch and a water-soluble polymer, an edible film comprising the same and preparation processes thereof.

### Advantageous Effects

The present edible film may not only have an excellent film forming property, solubility and feeling in the oral cavity, but also be easily prepared at low cost, and thus be usefully used as an oral cleanser or a bad breath remover for oral cleansing, and a soluble formulation on tongue for uptake of drug in the oral cavity.

### BEST MODE FOR CARRYING OUT THE INVENTION

The present invention provides an edible film composition comprising a waxy starch hydrolysate, a modified starch and a water-soluble polymer, an edible film comprising the same.

The waxy starch hydrolysate herein refers to a hydrolysate of a waxy starch. The waxy starch refers to a starch having higher amylopectin amount than an amylose amount, and refers to a starch comprising preferably 80% or more, more preferably 90% or more, of amylopectin. The waxy starch may be, but not limited to, a starch derived from one or more plants selected from the group consisting of, for example, rice, wheat, sweet potato, potato, corn and tapioca, and be easily obtained by genetic modification or hybridization.

Said waxy starch hydrolysate may be formed by treating a waxy starch with an acid or an enzyme. To have high aging stability, the oxidized, etherified or esterified starch may be also subjected to an acid or enzyme treatment, or the acid or enzyme treated waxy starch be subjected to oxidation, etherification or esterification as well.

Said acid treatment may be carried out by organic or inorganic acid treatment. For example, the waxy starch may be hydrolyzed by treating it with an organic acid such as acetic acid, formic acid, citric acid and malic acid, or an inorganic acid such as hydrochloric acid, sulfuric acid and phosphoric acid.

Said enzyme treatment may be carried out by one or more enzymes selected from the group consisting of, for example, alpha amylase, heat resistant alpha amylase, pullulanase, isoamylase, glucoamylase and beta amylase.

Conventionally, normal starches, waxy starches or high amylose starches have been used, if the starch is utilized as a film forming agent. However, the high amylose starches have excellent film formability, but a very low solubility in the oral cavity and organoleptic slipperiness, and the waxy starches have less organoleptic slipperiness, but remarkably lowered film formability. The normal starches have drawbacks that the film formabiltiy is lowered over the high amylose starches and the slipperiness is severe over the waxy starches. To complete such known drawbacks, in the present invention, hydrolysates of the waxy starches are utilized as a film forming agent. As can be identified from the following examples, the hydrolysates of the waxy starches have an excellent solubility in the oral cavity and no organoleptic slipperiness.

Preferably, the 20% solution of the waxy starch hydrolysate has a viscosity of 500 to 5,000 cps. Being lower or higher than the ranges, the film formability is lowered. The viscosity is measured by stirring 80% by weight of water and 20% by weight of the waxy starch hydrolysate, followed by using B type Brookfield viscometer at 40° C.

On the one hand, the modified starches refer to starches modified through one or more modification reactions such as oxidation, esterification, etherification, cross-linking reaction and pregelatinization, etc.

In one embodiment, said modified starch may be one or more starches selected from the group consisting of oxidized starch, acetylated distarch adipate, acetylated distarch phosphate, starch sodium octenyl succinate, starch octenyl succinate, octenyl distarch phosphate, monostarch phosphate, phosphated distarch phosphate, starch acetate, oxidized starch acetate, hydroxyethyl starch, hydroxypropyl distarch phosphate and hydroxypropyl starch. Preferably, said modified starch may be one or more starches selected from the group consisting of oxidized starch, starch sodium octenyl succinate, low viscosity starch octenyl succinate, oxidized starch acetate and hydroxypropyl starch.

In one embodiment, said modified starch may be a modified starch comprising less than 50% amylose. In another embodiment, said modified starch may be a modified starch comprising less than 40% amylose. In another embodiment, said modified starch may be a modified starch comprising less than 35% amylose. In other embodiment, said modified starch may be a modified starch comprising less than 30% amylose.

The present edible film composition also comprises a water-soluble polymer.

In one embodiment, said water-soluble polymer may be one or more water-soluble polymers selected from the group consisting of pullulan, gelatin, pectin, low viscosity pectin, hydroxypropylmethyl cellulose, low viscosity hydroxypropylmethyl cellulose, alginic acid, low viscosity alginic acid, sodium alginate, carrageenan, arabic gum, guar gum, locust bean gum, xanthan gum, gellan gum and agar. Preferably, said water-soluble polymer may be one or more water-soluble polymers selected from the group consisting of pullulan, gelatin, pectin, low viscosity pectin, low viscosity alginic acid, hydroxypropylmethyl cellulose and low viscosity hydroxypropylmethyl cellulose.

Waxy starch hydrolysates, modified starches and water-soluble polymers herein are included in said edible film composition without limitation about any part by weight, which may optionally vary with use of said edible film composition. For example, if the present edible film is used as a soluble formulation on tongue, the parts by weight of said components would be controlled such that they do not affect the strength of film, even though the amount of a drug increases. In one embodiment of the present invention, said edible film composition may comprise waxy starch hydrolysates, modified starches and water-soluble polymers in a ratio of 1 to 90:1 to 90:1 to 90 parts by weight. In other embodiment of the present invention, said edible film composition may comprise waxy starch hydrolysates, modified starches and water-soluble polymers in a ratio of 10 to 50:10 to 50:10 to 70 parts by weight.

In said edible film, waxy starch hydrolysates, modified starches and water-soluble polymers may be included in an amount of 40 to 99 parts by weight relative to the total weight of the edible film. Preferably, waxy starch hydrolysates, modified starches and water-soluble polymers may be included in an amount of 50 to 95 parts by weight relative to the total weight of the edible film.

The edible film composition of the present invention may further comprise one or more additives, in adition to waxy starch hydrolysates, modified starches and water-soluble polymers.

Said aditive may be fillers, plasticizers, sweeteners, acidulants, flavorings, emulsifiers, bad breath removers, colors, refrigerants, and the like.

Fillers play a role to reduce a characteristic that the film is slippery in the oral cavity and to give the film a skeleton. In ailition, they may reduce a property to be adhered between films, and control stickiness and decomposition rate of film and elution rate of drug in the oral cavity. Fillers may be ailed in an amount of 1 to 15 parts by weight relative to the total weight of the edible film composition.

In one embodiment, said filler may be one or more components selected from the group consisting of microcrystalline cellulose, cellulose polymers, magnesium carbonate, calcium carbonate, limestone powder, silicate, clay, talc, titanium dioxide and calcium phosphate.

Plasticizers may be used on controlling flexibility of films. Plasticizers may be ailed in an amount of 0.1 to 15 parts by weight relative to the total weight of the edible film composition.

In one embodiment, said plasticizer may be one or more components selected from the group consisting of sorbitol, maltitol, xylitol, glycerine, polyethylene glycol, propylene glycol, hydrogenated starch syrup, starch syrup, glycerine, triacetin, glycerol oleate, sucrose fatty acid esters and medium-chain fatty acids.

The edible film composition of the present invention may comprise sweeteners. Sweeteners may be ailed in an amount of 1 to 10 parts by weight relative to the total weight of the edible film composition.

In one embodiment, said sweetener may be one or more components selected from the group consisting of sugar, glucose, maltose, oligosaccharide, dextrin, invert sugar, fructose, lactose, galactose, starch syrup, sorbitol, maltitol, xylitol, erythritol, hydrogenated starch syrup, mannitol, trehalose, aspartame, acesulpame salts, neotame, sucralose, thaumatin, saccharin, licorice extract, stevioside, enzyme-treated stevioside, neohesperidin and monellin.

Also, the edible film composition of the present invention may further comprise an acidulant. The acidulant can play a role, together with the sweetener, in regulating taste and stimulating to generate saliva such that the edible film may be well dissolved. The acidulant may be ailed in an amount of 0.1 to 10 parts by weight relative to the total weight of the edible film composition.

In one embodiment, said acidulant may be one or more components selected from the group consisting of citric acid, malic acid, fumaric acid, tartaric acid, ascorbic acid, succinic acid, adipic acid and lactic acid.

The edible film composition of the present invention may further comprise a flavoring. The edible film of the present invention is required to all a suitable flavoring thereto, since it is a product which is dissolved and eaten in the oral cavity. Said flavoring may be a natural flavoring, an artificial flavoring or a mixture thereof. The natural flavoring may be extracts from plant leaves, flowers or fruits, plant oils, and the like. For example, fruit essence such as apple, peach, grape, strawberry, Rubus coreanus Miguel, raspberry, cherry, plum, citron, pineapple, apricot, and Chinese quince, and the like may be used as a natural flavoring. The plant oil includes spearmint oil, cinnamon oil, peppermint oil, lemon oil, clove oil, bay oil, thyme oil, cedar leaf oil, nutmeg oil, sage oil and almond oil. In adition, artificial synthetic fruit flavors such as lemon, orange, grape, lime and strawberry, and artificial synthetic flavors such as vanilla, chocolate, coffee, cocoa, pine needles, ginseng, red ginseng and citrus may be used as an artificial flavoring. The amount of the flavoring varies with various factors such as types, kinds and the desired intensity of usually used flavorings, which may be, generally, 1 to 15 parts by weight relative to the total weight of the edible film composition.

Oil type flavorings may be used together with an emulsifier, to be incorporated with water-soluble materials. The amount of the emulsifier may be regulated depending on kinds and quantities of flavorings. Generally, it may be ailed in an amount of 0.1 to 10 parts by weight relative to the total weight of the edible film composition.

In one embodiment, said emulsifier may be one or more components selected from the group consisting of glycerin fatty acid esters, sucrose fatty acid esters, lecithin, enzyme-treated lecithin, polysorbates, sorbitan fatty acid esters and propylene glycol.

In the edible film composition of the present invention, a bad breath remover for alleviating bad breath may be further included, in ailition to said flavoring. The amount of the bad breath remover may be regulated. Generally, it may be ailed in an amount of 0.1 to 10 parts by weight relative to the total weight of the edible film composition. In one embodiment, said bad breath remover may be metal salts. Said metal salts may be one or more components selected from the group consisting of metal chlorites, copper gluconate, zinc chloride, zinc citrate and zinc gluconate. In another embodiment, said bad breath remover may be one or more components selected from the group consisting of triclosan, alexidine, hexetidine, benzalkonium chloride, salicylanilide, domiphen bromide, tetradecylpyridinium chloride, N-tetradecyl-4-ethylpyrilinium chloride, octenidine, iodine, sulfonamide, bisbiguanile, phenols, delmopinol, octapinol, chlorhexidine, nisin formulations, nystatin, sanguinarine, cetylpyridinium chloride, red ginseng extracts, green tea extracts, seaweed extracts, herb extracts, grapefruit extracts, apple extracts, thyme oil, thymol, antibiotics, geraniol, carvacrol, citral, hinokitiol, ucalyptol, catechol, methylsalicylate and hydrogen peroxide. At least one of such components for bad breath removers may be used, independently or together with one or more of said metal salts.

In addition, the edible film composition of the present invention may comprise colors appropriate to products. If necessary, the amount of the colors may be suitably regulated. Generally, it may be ailed in an amount of 0.01 to 10 parts by weight relative to the total weight of the edible film composition. Said colors may be natural or synthetic colors.

Also, the edible film composition of the present invention may further comprise refrigerants. The refrigerants may be, but not limited to, for example, WS3, WS23 or Questice-L. If necessary, the amount of the refrigerant may be suitably regulated. Generally, it may be ailed in an amount of 0.01 to 5 parts by weight relative to the total weight of the edible film composition.

The present edible film may be not only used as an oral cleanser or a bad breath remover, but also utilized as a soluble formulation on tongue.

Therefore, the present invention also provides a soluble formulation on tongue comprising a waxy starch hydrolysate, a modified starch, a water-soluble polymer and a pharmaceutically active ingredient.

The pharmaceutically active ingredient may includes, but is not limited to, for example, antidiabetics such as glimepiride and pioglitazone; insomnia therapeutics such as zolpidem and eszopiclone; urogenital therapeutics such as tolteroiine and trospium; obesity therapeutics such as sibutramine; enzymes such as streptokinase; anti-peptic ulcer agents such as omeprazole; antitussives/expectorants such as clenbuterol hydrochloride; skin disorder therapeutics such as finasteride; antiemetics such as ondansetron; antidepressants such as fluoxetin; antihistamines such as fexofenadine hydrochloride; antipyretic analgesics/anti-immflammatory agents such as asprin, ibuprofen and meloxicam; hormones such as testosterone; circulatory therapeutics such as fleodipine, atrovastatin, amlodipine camsylate, doxazosin, simvastatin and lercanidipine; digestive organ therapeutics such as famotidine and lansoprazole; psychoneurosis therapeutics such as paroxetine; impotence therapeutics such as sildenafil and tadalafil; osteoporosis therapeutics; arthritis therapeutics; epilepsy therapeutics; muscle relaxants; brain function improvers; schizophrenia therapeutics; immunosuppressants; antibiotics; anticancer drugs; anticancer therapeutic supplements; vaccines; oral cleansers; antianemics; constipation therapeutics; vitamins; nutrients; lactobacillus formulations; anti-common-cold drug complexes; or health care foals, and the like.

Any kind of pharmaceutically active ingredient, which may utilize the present edible film as a drug delivery system, does not be restricted. If it is any drug which can be orally administrated through the oral cavity, it may be used. For example, said pharmaceutically active ingredient may be one or more components selected from the group consisting of triclosan, cetyl pyridiumchloride, domiphen bromide, quaternary ammonium salts, zinc compounds, sanguinarine, fluorides, alexidine, octonidine, EDTA, aspirin, acetaminophen, ibuprofen, ketoprofen, diflunisal, fenoprofen calcium, naproxen, tolmetin sodium, indomethacin, benzonatate, caramiphen, edisylate, menthol, dextromethorphan, hydrobromides, hydrochlorides, chlophedianol, diphenhydramine, pseudoephedrine, phenylepherine, phenylpropanolamine, pseudoephedrine sulfate, brompheniramine maleate, chlorpheniramine maleate, carbinoxamine maleate, clemastine fumarate, dexchlorpheniramine maleate, diphenhydramine hydrochloride, diphenhydramine citrate, diphenylpyraline hydrochloride, doxylamine succinate, promethazine hydrochloride, pyrilamine maleate, tripelenamine citrate, triprolidine hydrochloride, acrivastine, loratadine, brompheniramine, dexbrompheniramine, guaifenesin, ipecac, calcium iodide, terpin hydrate, loperamide, famotidine, ranitidine, omeprazole, lansoprazole, aliphatic alcohols, nicotine, caffeine, strychnine, picrotoxin, pentylenetetrazol, phenyhydantoin, phenobarbital, primidone, carbamazepine, ethosuximide, methsuximide, phensuximide, trimethadione, diazepam, benzodiazepine, phenacemide, pheneturide, acetazolamide, sulthiame, bromides, levodopa, amantadine, morphine, heroin, hydromorphone, metopon, oxymorphone, levorphanol, codeine, hydrocodone, xycodone, nalorphine, naloxone, naltrexone, salycilates, phenylbutazone, indomethacin, phenacetin, chlorpromazine, methotrimeprazine, haloperidol, clozapine, reserpine, imipramine, tranylcypromine, phenelzine, lithium, apomorphine, sildenafil, tadalafil, vardenafil, zolpidem tartrate, bambuterol hydrochloride, galantamine chlorobromide, nicardipine hydrochloride, paroxetine hydrochloride, meloxicam, tolteridine tartrate and cbxazosin mesylate.

The present invention also provides an edible film comprising said edible film composition. Preferably, the present edible film will be formed as a thin film maintaining a suitable range of tensile strength and toughness under the condition of a very thin film. In one embodiment, the present edible film has a thickness of 20 μm to 100 μm and preferably, 40 μm to 60 μm If the thickness is in excess of 100 μm organoleptic slipperiness increases, which can be somewhat complemented by regulating contents of sweeteners, acidulants, gelling agents, fillers, flavorings, and the like. However, if it is too thick, its melting time increases and thus, the organoleptic slipperiness is not easily overcome.

The present invention also provides a process for preparing an edible film.

In one embodiment, the process for preparing the edible film of the present invention may comprise

(1) preparing an edible film composition comprising a waxy starch hydrolysate, a modified starch and a water-soluble polymer;

(2) pouring said edible film composition into a molding machine to mold it into a film at 50 to 80° C.;

(3) maturing said molded film at a relative humidity of 50 to 70% for 1 to 10 days.

For example, the process for preparing an edible film according to the present invention may be carried out via the following processes.

(1) Solutions Preparation Process

a) Process for preparing a first solution: A waxy starch hydrolysate, a modified starch and a water-soluble polymer are poured into boiling water, respectively, to be completely dissolved therein.

b) Process for preparing a second solution: additives such as colors, sweeteners, acidulants, fillers and the likes are mixed to prepare the second solution.

c) Process for preparing a third solution: mentol, a flavoring and a refrigerant are mixed with an emulsifier to prepare the third solution.

d) Process for preparing a fourth solution: said second solution is mixed with said third solution to prepare the fourth solution.

e) Process for preparing a fifth solution: with increasing the temperature to 60° C. or more, said first solution is mixed with said fourth solution to prepare the fifth solution.

(2) Molding Process

Said fifth solution is filtered off and poured into a molding machine to mold it into a film. At this time, the temperature of said molding machine is 50 to 80° C., and it is prepared in a form of roll via a belt drum dryer.

(3) Maturing Process

The molded film is subjected to a maturing process in a relative humidity of 50 to 70% for 1 to 10 days to contain moisture suitable for slitting and cutting it. At this time, the moisture content is suitably 10 to 14%.

As subsequent processes of said processes, the following processes may be further carried out.

(4) Cutting Process

The matured roll is slit to small rolls, cut in a suitable size, and filled in a small container.

(5) Packaging Process

The filled product in the small container is labeled, and then finished via repacking or blister packing thereon.

### MODE FOR THE INVENTION

### Examples

Experimental Example 1

### Evaluation of Film Forming Ability, Taste and Solubility according to Kinds of Starches

To identify superiority of waxy starch hydrolysates, edible films were prepared in compositions of Table 1 below, with changing kinds of starches into high amylose corn starch (A), corn starch (B), waxy corn starch (C), and waxy corn starch hydrolysate (D), to evaluate the film forming ability, taste and solubility thereof.

As modified starches, oxidized starch, oxidized starch acetate, hydroxypropyl corn strach (HP starch), and low viscosity starch octenyl succinate (low viscosity OS starch) were used.

Amylogel 03001 manufactured by Cargill, Inc. was used as the high amylose corn starch (A), and corn starch, sticky corn starch and BTR sticky starch manufactured by Daesang Corporation (KR) were used as corn starch (B), waxy corn starch (C) and waxy corn starch hydrolysate (D), respectively. The low viscosity OS starch was used by reacting the OS starch (trade name: HITEX) obtained from Daesang Corporation with 1N hydrochloric acid for 1 hour to reduce viscosity.

The HP starch was used by reacting Kreation D8 obtained from AVEBE with 1N hydrochloric acid for 1 hour to reduce viscosity. The oxidized starch acetate was utilized from the oxidized starch manufactured by Daesang Corporation, by reacting it with acetic anhydride. At this time, the substitution degree of acetyl group was 3% (containing 3 acetyl groups per 100 molecules of glucose). As the low viscosity HPMC, Demacol HE 5/6 BV from Demacsa was used. As the low viscosity pectin, GENU Pectin DSS from CP Kelco was used. As the low viscosity alginic acid, Login from MSC Cb., Ltd. (KR) was used. As gelatin, a product having strength of 100Bloom from Geltech Cb., Ltd. (KR) was used, and as pullulan, a product from Shanghai Puao Biotech Cb., Ltd. (CN) was used.

Table 1

TABLE 1
Film Formability, taste and solubility according to kind of starch

| | | | Modified | | Water-soluble | | | | |
|---|---|---|---|---|---|---|---|---|---|
| | Starch | | Starch | | Polymer | | | Feeling | |
| | | AMOUNT | | AMOUNT | | AMOUNT | Film | in oral | SOLUBILITY |
| COMBINATION | KIND | (%) | Kind | (%) | Kind | (%) | Formability | cavity | (s) |
| 1 | A | 10 | Oxidized Starch | 20 | LV Alginic | 15 | H | L | 21 |
| | | | Acetate | | Acid | | | | |
| 2 | B | 8 | Oxidized Starch | 20 | LV Alginic | 15 | M | M | 16 |
| | | | Acetate | | Acid | | | | |
| 3 | C | 15 | Oxidized Starch | 20 | LV Alginic | 15 | L | SM | 11 |
| | | | Acetate | | Acid | | | | |
| 4 | D | 25 | Oxidized Starch | 20 | LV Alginic | 15 | M | H | 10 |
| | | | Acetate | | Acid | | | | |
| 5 | A | 10 | HP Starch | 20 | LV Pectin | 13 | H | L | 19 |
| 6 | B | 8 | HP Starch | 20 | LV Pectin | 13 | M | M | 14 |
| 7 | C | 15 | HP Starch | 20 | LV Pectin | 13 | L | M | 10 |
| 8 | D | 25 | HP Starch | 20 | LV Pectin | 13 | M | SM | 10 |
| 9 | A | 10 | LV OS Starch | 20 | Gelatin | 25 | H | L | 16 |
| 10 | B | 8 | LV OS Starch | 20 | Gelatin | 25 | M | M | 13 |
| 11 | C | 15 | LV OS Starch | 20 | Gelatin | 25 | L | H | 8 |
| 12 | D | 25 | LV OS Starch | 20 | Gelatin | 25 | M | H | 7 |
| 13 | A | 10 | HP Starch | 20 | Pullulan | 22 | H | L | 16 |
| 14 | B | 8 | HP Starch | 20 | Pullulan | 22 | M | M | 12 |
| 15 | C | 15 | HP Starch | 20 | Pullulan | 22 | L | H | 8 |
| 16 | D | 25 | HP Starch | 20 | Pullulan | 22 | M | H | 7 |
| 17 | A | 10 | Oxidized Starch | 20 | LV HPMC | 20 | H | L | 25 |
| 18 | B | 8 | Oxidized Starch | 20 | LV HPMC | 20 | M | M | 16 |
| 19 | C | 15 | Oxidized Starch | 20 | LV HPMC | 20 | L | SM | 11 |
| 20 | D | 25 | Oxidized Starch | 20 | LV HPMC | 20 | M | H | 10 |

* Contents of starches, modified starches and water-soluble polymers are based on added solid contents in total solid contents (wt/wt).
Notes:
LV = Low Viscosity,
H = High,
M = Medial,
L = Low,
SM = Super-medial

As can be seen from Table 1, it shows excellent results in aspects of film forming ability, taste and solubility, to use waxy starch hydrolysate together with modified starch and water-soluble polymer over to use high amylose starch, normal starch or waxy starch.

### Preparation Example 1

According to the following method, components were introduced as their ratios in Table 2 to prepare an edible film.

(1) Water was heated to 90° C. or more, and a waxy starch hydrolysate, hydroxypropyl starch and pectin were ailed thereto to prepare a 10% solution. The gelatinized liquid was cooled to 70° C.

(2) A color, a high sweet sweetener, citric acid, and microcrystalline cellulose were mixed with said first step solution to prepare a second step solution.

(3) Mentol, a flavoring and an emulsifier were dissolved and emulsified in water at 60° C. to prepare a third step solution.

(4) Said second step solution was mixed with said third step solution to prepare a fourth step solution. After filtering off said fourth step solution, it was poured into a molding machine to mold it into a film, followed by maturing it at a relative humility of 60% for 7 days as such and packaging the product in a container via slitting and cutting.

TABLE 2

| | Preparation | Comparative | | |
|---|---|---|---|---|
| | Example | Example | | |
| | (wt/wt %) | (wt/wt %) | | |
| Components | 1 | 1 | 2 | 3 |
| Waxy Starch | 36.8 | | | |
| Hydrolysate | | | | |
| High Amylose | | 18.9 | | |
| Corn Starch | | | | |
| Corn Starch | | | 15.7 | |
| Waxy Corn Starch | | | | 25.9 |
| Hydroxypropyl | 29.4 | 37.7 | 39.2 | 34.5 |
| Starch | | | | |
| Pectin | 7.4 | 9.4 | 9.8 | 8.6 |
| Microcrystalline | 7.5 | 9.6 | 10.0 | 8.8 |
| Cellulose | | | | |
| Sucralose | 1.5 | 1.9 | 2.0 | 1.7 |
| Aspartame | 1.5 | 1.9 | 2.0 | 1.7 |
| Licorice Extract | 1.5 | 1.9 | 2.0 | 1.7 |
| Citric Acid | 0.9 | 1.1 | 1.2 | 1.0 |
| Mentol | 5.1 | 6.6 | 6.9 | 6.0 |
| Coffee Flavor | 5.7 | 7.4 | 7.6 | 6.7 |
| Polysorbate | 0.9 | 1.1 | 1.2 | 1.0 |
| Glycerine | 0.9 | 1.1 | 1.2 | 1.0 |
| WS-3 | 0.7 | 0.9 | 1.0 | 0.9 |
| Blue No. 1 | 0.1 | 0.2 | 0.2 | 0.2 |
| Eucalyptol | 0.1 | 0.2 | 0.2 | 0.2 |
| Total | 100.0 | 100.0 | 100.0 | 100.0 |

* Input calculated by estimating moisture to be 10% after preparing a film

### Comparative Preparation Example 1

This example was carried out by the same method as Preparation Example 1 except that high amylose starch was introduced instead of waxy starch hydrolysate.

### Comparative Preparation Example 2

This example was carried out by the same method as Preparation Example 1 except that corn starch was introduced instead of waxy starch hydrolysate.

### Comparative Preparation Example 3

This example was carried out by the same method as Preparation Example 1 except that waxy starch was introduced instead of waxy starch hydrolysate.

### Experimental Example 2

For the edible films of Preparation Example 1 and Cbmparative Prepareation Examples 1 to 3, film forming ability, film solubility and organoleptic slipperiness were compared and evaluated. The films had good film forming ability in the order of Comparative Preparation Example 1>Comparative Preparation Example 2>Comparative Preparation Example 3=Preparation Example 1, and good film solubility and organoleptic slipperiness in the order of Preparation Example 1>Comparative Preparation Example 3>Comparative Preparation Example 2>Comparative Preparation Example 1.

### Preparation Examples 2 to 5

Contrary to using hydroxypropyl starch as the malified starch in Preparation Example 1, edible films were prepared as compositions in Table 3 by using oxidized starch acetate, oxidized starch and starch sodium octenyl succinate as the modified starch.

On utilizing oxidized starch acetate, oxidized starch and starch sodium octenyl succinate, the films did not show much difference in film forming ability, film solubility and organoleptic slipperiness, except for convenience in use.

TABLE 3

| | Preparation Example (wt/wt %) | | | |
|---|---|---|---|---|
| Components | 2 | 3 | 4 | 5 |
| Waxy Starch Hydrolysate | 34.2 | 34.2 | 34.2 | 34.2 |
| Hydroxypropyl Starch | 27.4 | | | |
| Oxidized Starch Acetate | | 27.4 | | |
| Oxidized Starch | | | 27.4 | |
| Starch Sodium Octenyl Succinate | | | | 27.4 |
| Pectin | 13.7 | 13.7 | 13.7 | 13.7 |
| Microcrystalline Cellulose | 7.0 | 7.0 | 7.0 | 7.0 |
| Sucralose | 1.4 | 1.4 | 1.4 | 1.4 |
| Aspartame | 1.4 | 1.4 | 1.4 | 1.4 |
| Licorice Extract | 1.4 | 1.4 | 1.4 | 1.4 |
| Citric Acid | 0.8 | 0.8 | 0.8 | 0.8 |
| Mentol | 5.0 | 5.0 | 5.0 | 5.0 |
| Lemon Flavor | 5.3 | 5.3 | 5.3 | 5.3 |
| Polysorbate | 0.8 | 0.8 | 0.8 | 0.8 |
| Glycerine | 0.8 | 0.8 | 0.8 | 0.8 |
| WS-3 | 0.7 | 0.7 | 0.7 | 0.7 |
| Yellow No. 4 | 0.1 | 0.1 | 0.1 | 0.1 |
| Total | 100.0 | 100.0 | 100.0 | 100.0 |

* Input calculated by estimating moisture to be 10% after preparing a film

### Preparation Examples 6 to 9

Edible films were prepared as combination ratios in Table 4, by using gelatin, pullulan and hydroxypropylmethyl cellulose as a water-soluble polymer, instead of pectin.

TABLE 4

| | Preparation Example (wt/wt %) | | | |
|---|---|---|---|---|
| Components | 6 | 7 | 8 | 9 |
| Waxy Starch Hydrolysate | 33.3 | 28.4 | 29.4 | 28.4 |
| Hydroxypropyl Starch | 26.7 | 22.7 | | |
| Oxidized Starch | | | 23.5 | 22.7 |
| Pectin | 16.0 | | | |
| Gelatin | | 28.4 | | |
| Pullulan | | | 25.9 | |
| Hydroxypropylmethyl Cellulose | | | | 28.4 |
| Microcrystalline Cellulose | 6.8 | 5.8 | 6.0 | 5.8 |
| Sucralose | 1.3 | 1.1 | 1.2 | 1.1 |
| Aspartame | 1.3 | 1.1 | 1.2 | 1.1 |
| Licorice Extract | 1.3 | 1.1 | 1.2 | 1.1 |
| Citric Acid | 0.8 | 0.7 | 0.7 | 0.7 |
| Mentol | 4.7 | 4.0 | 4.1 | 4.0 |
| Grape Flavor | 5.2 | 4.4 | 4.6 | 4.4 |
| Polysorbate | 0.8 | 0.7 | 0.7 | 0.7 |
| Glycerine | 0.8 | 0.7 | 0.7 | 0.7 |
| WS-3 | 0.7 | 0.6 | 0.6 | 0.6 |
| Cochineal Extract Color | 0.1 | 0.1 | 0.1 | 0.1 |
| Eucalyptol | 0.1 | 0.1 | 0.1 | 0.1 |
| Total | 100.0 | 100.0 | 100.0 | 100.0 |

* Input calculated by estimating moisture to be 10% after preparing a film

On evaluating film forming ability, film solubility and organoleptic slipperiness, the films had good film forming ability in the order of gelatin>pullulan>hydroxypropylmethyl cellulose>pectin. Also, they had good film solubility and organoleptic slipperiness in the order of gelatin>pullulan>hydroxypropylmethyl cellulose>pectin.

### Preparation Examples 10 to 14

Films were prepared as compositions in Table 5 by ailing pharmaceutically active ingredients including melatonin and zolpidem tartrate.

TABLE 5

| | Preparation Example (wt/wt %) | | | | |
|---|---|---|---|---|---|
| Components | 10 | 11 | 12 | 13 | 14 |
| Waxy Starch Hydrolysate | 25.0 | 22.1 | 25.6 | 25.7 | 27.5 |
| Hydroxypropyl Starch | 20.0 | 17.7 | | | |
| Oxidized Starch | | | 20.6 | 20.7 | 21.9 |
| Hydroxypropylmethyl Cellulose | 22.0 | | | | 24.3 |
| Gelatin | | 19.4 | | 22.7 | |
| Pullulan | | | 22.7 | | |
| Microcrystalline Cellulose | 5.2 | 4.6 | 5.3 | 5.4 | 5.7 |
| Sucralose | 1.0 | 0.9 | 1.0 | 1.0 | 1.1 |
| Aspartame | 1.0 | 0.9 | 1.0 | 1.0 | 1.1 |
| Licorice Extract | 1.0 | 0.9 | 1.0 | 1.0 | 1.1 |
| Citric Acid | 0.6 | 0.5 | 0.6 | 0.6 | 0.7 |
| Mentol | 3.5 | 3.1 | 3.6 | 3.6 | 3.9 |
| Strawberry Flavor | 3.9 | 3.4 | 4.0 | 4.0 | 4.3 |
| Polysorbate | 0.6 | 0.5 | 0.6 | 0.6 | 0.7 |
| Glycerine | 0.6 | 0.5 | 0.6 | 0.6 | 0.7 |
| WS-3 | 0.5 | 0.4 | 0.5 | 0.5 | 0.6 |
| Blue No. 1 | 0.1 | 0.1 | 0.1 | 0.1 | 0.1 |
| Melatonin | 15.0 | | | | |
| Zolpidem Tartrate | | 25.0 | | | |
| Galantamine Chlorobromide | | | 12.8 | | |
| Tadalafil | | | | 12.5 | |
| Vardenafil | | | | | 6.3 |
| Total | 100.0 | 100.0 | 100.0 | 100.0 | 100.0 |

* Input calculated by estimating moisture to be 10% after preparing a film

On evaluating film forming ability, film solubility and organoleptic slipperiness, the films had no problem, when the pharmaceutically active ingredients were introduced in an amount of less than 20%. However, on introducing them in an amount of 30% or more, the films showed phenomena that film forming ability and strength were lowered.

1-11. (canceled)

12. An edible film made by an edible film composition which comprises a waxy starch hydrolysate, a modified starch and a water-soluble polymer.

13. The edible film according to claim 12,
wherein the waxy starch hydrolysate is a hydrolysate from a waxy starch comprising 80% or more of amylopectin.

14. The edible film according to claim 12,
wherein the waxy starch is a starch derived from one or more plants selected from the group consisting of rice, wheat, sweet potato, potato, corn and tapioca.

15. The edible film according to claim 12,
wherein the waxy starch hydrolysate is formed by treating a waxy starch with an acid or an enzyme.

16. The edible film according to claim 12,
wherein the waxy starch hydrolysate is formed by treating the oxidized, etherified or esterified starch with an acid or an enzyme or by oxydizing, etherifying or esterifying an acid- or enzyme-treated waxy starch.

17. (canceled)

18. The edible film according to claim 15,
wherein the enzyme treatment is carried out by one or more enzymes selected from the group consisting of alpha amylase, heat resistant alpha amylase, pullulanase, isoamylase, glucoamylase and beta amylase.

19. The edible film according to claim 12,
wherein the waxy starch hydrolysate has a viscosity, in 20% solution at 40° C., of 500 to 5,000 cps.

20. The edible film according to claim 12,
wherein the modified starch is modified from a starch by one or more reactions selected from the group consisting of oxidation, esterification, etherification, cross-linking reaction, and pregelatinization.

21. The edible film according to claim 12,
wherein the modified starch is one or more starches selected from the group consisting of oxidized starch, acetylated distarch adipate, acetylated distarch phosphate, starch sodium octenyl succinate, low viscosity starch octenyl succinate, octenyl distarch phosphate, monostarch phosphate, phosphated distarch phosphate, starch acetate, hydroxyethyl starch, oxidized starch acetate, hydroxypropyl distarch phosphate and hydroxypropyl starch.

22. (canceled)

23. The edible film according to claim 12,
wherein the modified starch is a modified starch comprising less than 50% amylose.

24. (canceled)

25. The edible film according to claim 12,
wherein the water-soluble polymer is one or more water-soluble polymers selected from the group consisting of pullulan, gelatin, pectin, low viscosity pectin, hydroxypropylmethyl cellulose, low viscosity hydroxypropylmethyl cellulose, alginic acid, low viscosity alginic acid, sodium alginate, carrageenan, arabic gum, guar gum, locust bean gum, xanthan gum, gellan gum and agar.

26. The edible film according to claim 12,
wherein the water-soluble polymer is one or more water-soluble polymers selected from the roup consisting of pullulan, gelatin, pectin, low viscosity pectin, low viscosity alginic acid, hydroxypropylmethyl cellulose and low viscosity hydroxypropylmethyl cellulose.

27. (canceled)

28. The edible film according to claim 12,
comprising 10 to 50 parts by weight of the waxy starch hydrolysate, 10 to 50 parts by weight of the modified starch and 10 to 70 parts by weight of the water-soluble polymer.

29. The edible film according to claim 12,
further comprising one or more additives selected from the group consisting of a filler, a plasticizer, a sweetener, an acidulant, a flavoring, an emulsifier, a bad breath remover, a color and a refrigerant.

30-37. (canceled)

38. The edible film according to claim 12,
wherein the edible film has a thickness of 20 μm to 100 μm.

39. A soluble formulation on tongue comprising a waxy starch hydrolysate, a modified starch, a water-soluble polymer and a pharmaceutically active ingredient.

40. The soluble formulation on tongue according to claim 39, wherein the pharmaceutically active ingredient is one or more ingredients selected from the group consisting of antidiabetics; insomnia therapeutics; urogenital therapeutics; obesity therapeutics; enzymes; anti-peptic ulcer agents; antitussives/expectorants; skin disorder therapeutics; antiemetics; antidepressants; antihistamines; antipyretic analgesics/anti-immflammatory agents; hormones; circulatory therapeutics; digestive organ therapeutics; psychoneurosis therapeutics; impotence therapeutics; osteoporosis therapeutics; arthritis therapeutics; epilepsy therapeutics; muscle relaxants; brain function improvers; schizophrenia therapeutics; immunosuppressants; antibiotics; anticancer drugs; anticancer therapeutic supplements; vaccines; oral cleansers; antianemics; constipation therapeutics; vitamins; nutrients; lactobacillus formulations; anti-common-cold drug complexes; or health care foods.

41. The soluble formulation on tongue according to claim 39, wherein the pharmaceutically active ingredient is one or more components selected from the group consisting of triclosan, cetyl pyridiumchloride, domiphen bromide, quarternary ammonium salts, zinc compounds, sanguinarine, fluorides, alexidine, octonidine, EDTA, aspirin, acetaminophen, ibuprofen, ketoprofen, diflunisal, fenoprofen calcium, naproxen, tolmetin sodium, indomethacin, benzonatate, caramiphen, edisylate, menthol, dextromethrophan, hydrobromides, hydrochlorides, chlophedianol, diphenhydramine, pseudoephedrine, phenylepherine, phenylpropanolamine, pseudoephedrine sulfate, brompheniramine maleate, chlorpheniramine maleate, carbinoxamine maleate, clemastine fumarate, dexchlorpheniramine maleate, diphenhydramine hydrochloride, diphenhydramine citrate, diphenylpyraline hydrochloride, doxylamine succinate, promethazine hydrochloride, pyrilamine maleate, tripelenamine citrate, triprolidine hydrochloride, acrivastine, loratadine, brompheniramine, dexbrompheniramine, guaifenesin, ipecac, calcium iodide, terpin hydrate, loperamide, famotidine, ranitidine, omeprazole, lansoprazole, aliphatic alcohols, nicotine, caffeine, strychnine, picrotoxin, pentylenetetrazol, phenyhydantoin, phenobarbital, primidone, carbamazepine, ethosuximide, methsuximide, phensuximide, trimethadione, diazepam, benzodiazepine, phenacemide, pheneturide, acetazolamide, sulthiame, bromides, levodopa, amantadine, morphine, heroin, hydromorphone, metopon, oxymorphone, levorphanol, codeine, hydrocodone, xycodone, nalorphine, naloxone, naltrexone, salycilates, phenylbutazone, indomethacin, phenacetin, chlorpromazine, methotrimeprazine, haloperidol, clozapine, reserpine, imipramine, tranylcypromine, phenelzine, lithium, apomorphine, sildenafil, tadalafil, vardenafil, zolpidem tartrate, bambuterol hydrochloride, galantamine chlorobromide, nicardipine hydrochloride, paroxetine hydrochloride, meloxicam, tolteridine tartrate and doxazosin mesylate.

42. A process for preparing an edible film comprising:
(1) preparing an edible film composition comprising a waxy starch hydrolysate, a modified starch and a water-soluble polymer;
(2) pouring the edible film composition into a molding machine to mold it into a film at 50 to 80° C.;
(3) maturing the molded film at a relative humidity of 50 to 70% for 1 to 10 days.
